(19) United States
(12) Patent Application Publication
(10) Pub. No.: US 2014/0213924 A1
(43) Pub. Date: Jul. 31, 2014

(54) METHODS, SYSTEMS AND COMPUTER READABLE STORAGE MEDIA STORING INSTRUCTIONS FOR DETERMINING RESPIRATORY INDUCED ORGAN MOTION

(71) Applicants: Mohammad K. Khan, Atlanta, GA (US); Tian Liu, Atlanta, GA (US); Yuting Lin, Cambridge, MA (US)

(72) Inventors: Mohammad K. Khan, Atlanta, GA (US); Tian Liu, Atlanta, GA (US); Yuting Lin, Cambridge, MA (US)

(73) Assignee: EMORY UNIVERSITY, Atlanta, GA (US)

(21) Appl. No.: 14/165,771

(22) Filed: Jan. 28, 2014

Related U.S. Application Data
(60) Provisional application No. 61/757,486, filed on Jan. 28, 2013.

(51) Int. Cl. A61B 5/113

(52) U.S. Cl. CPC A61B 5/113
USPC 600/534

(57) ABSTRACT

Methods, systems and computer-readable storage media relate to determining respiratory motion from motion data of a target to be treated. The methods may include processing motion data of a target to be treated obtained from at least one marker for at least one period. Each period including a plurality of time intervals. The processing including processing the motion data to determine an isocenter for each time interval along at least one of a plurality of axes of motion. The method may include determining at least one component of the motion data in at least one axis, the at least component corresponding to a subset of the motion data having a discrete value and/or a range of values; and determining respiratory motion from at least one component of the motion data. Radiotherapy treatment can be improved by determining the respiratory motion and the impact of respiration of a target.

### CROSS REFERENCE TO RELATED APPLICATION

This application claims priority to Provisional Application Ser. No. 61,757,486 filed Jan. 28, 2013, which is hereby incorporated by reference in its entirety.

### BACKGROUND

Prostate cancer is one of the leading cancers diagnosed in men within the United States and is expected to affect 241,470 men of which 28,000 will die in 2012. See, e.g., Siegel R, Ward E, Brawley O, et al. Cancer statistics, 2011: the impact of eliminating socioeconomic and racial disparities on premature cancer deaths. CA Cancer J Clin; 61:212-236. One of the variety of treatment options includes radiotherapy.

Organ motion during radiotherapy treatment can contribute to the degrading of the dose distribution and increase toxicity of surrounding tissue. For example, for the prostate, the prostate motion can generally result from the bladder and rectum filling and respiratory motion. A conventional way to address possible organ motion in radiotherapy is to surround the clinical target volume (CTV) with a margin to allow for setup uncertainties and movement. This margin should be as small as possible as it increases the volume of normal tissue irradiated and thereby can increase the potential for short-term and long-term side effects to the surrounding tissue. However, many of the current margin models generally address possible organ motion and do not consider the contribution of respiratory motion.

### SUMMARY

Thus, there is a need for determining respiratory motion from intrafraction organ motion (e.g., a prostate).

The disclosure relates to systems, methods, and computer-readable media storing instructions for determining respiratory motion from motion data of a target to be treated. In this way, respiratory induced impact on target motion may be separated from background noise and other physiological parameters.

In some embodiments, the methods may relate to a method of determining an impact of respiratory motion with respect to movement of a target to be treated. In some embodiments, the method may include processing motion data of a target obtained from at least one marker for at least one period. Each period may include a plurality of time intervals, the processing including processing the motion data to determine an isocenter for each time interval along at least one of a plurality of axes of motion. The plurality of axes may include x axis, y axis, and/or z axis. In some embodiments, the method may include determining at least one component of the processed motion data in at least one axis, the at least component corresponding to a subset of the motion data having a discrete value and/or a range of values. In some embodiments, the method may include determining respiratory motion from at least one component of the motion data. The determining the at least one component includes applying at least one threshold to the processed motion data. In some embodiments, the motion data may be obtained in substantially real-time. In some embodiments, the threshold corresponds to a range of frequencies associated with a respiratory pattern, for example, a range of frequencies of about 0.166-0.4 Hz. The methods can be performed by a computer having a memory and a processor. The methods may also be performed automatically.

In some embodiments, the systems may relate to a system for determining respiratory motion. The system may include a motion data processor configured to process motion data of a target obtained from at least one marker for at least one period. Each period may include a plurality of time intervals. The processing may include processing the motion data to determine an isocenter for each time interval along at least one of a plurality of axes of motion. The plurality of axes may include x axis, y axis, and/or z axis. In some embodiments, the system may include a respiratory motion determination module configured to determine at least one component of the motion data in at least one axis, the at least component corresponding to a subset of the motion data having a discrete value and/or a range of values and configured to respiratory motion from at least one component of the motion data.

In some embodiments, the computer readable media may relate to a computer-readable medium storing instructions for determining respiratory motion. The instructions may include processing motion data of a target obtained from at least one marker for at least one. Each period may include a plurality of time intervals, the processing including processing the motion data to determine an isocenter for each time interval along at least one of a plurality of axes of motion. The plurality of axes may include x axis, y axis, and/or z axis. In some embodiments, the instructions may include determining at least one component of the processed motion data in at least one axis, the at least component corresponding to a subset of the processed motion data having a discrete value and/or a range of values. In some embodiments, the instructions may include determining respiratory motion from at least one component of the motion data. The computer readable storage medium may be a non-transitory medium.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows a block diagram illustrating a system according to embodiments;

FIG. 2 shows a block diagram illustrating an example of a computing system;

FIG. 3 shows a method of determining respiratory motion from motion data of a target according to embodiments;

FIG. 4 shows a method of processing motion data according to embodiments;

FIG. 5 shows an example of raw motion data received from an electromagnetic transponder;

FIG. 6 shows a diagram of position data for a time point obtained from a marker or a transponder implanted into a target;

FIG. 7 shows a method of determining respiratory motion according to embodiments;

FIG. 8 shows an example of determining at least one component of motion data according to embodiments;

FIGS. 9(a) and 9(b) shows an example of motion data of a target obtained from a transponder;

FIG. 10 shows an example of a filtered motion data into a plurality of components;

FIG. 11 shows an example of respiratory motion parameters determined from the components of FIG. 10; and

FIG. 12 shows a method of adjusting and/or determining treatment based on respiratory motion according to embodiments;

### DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed methods, systems, and computer-readable media relate to determining The disclosed methods, systems, and computer-readable media according to embodiments can determine respiratory motion in at least axis based on motion data of a target (e.g., an organ). The disclosed methods, systems, and computer-readable media according to embodiments can improve radiotherapy treatment by determining the respiratory motion and the impact of respiration of an organ. Treatment margins can be smaller and more optimal because the delivery of the treatment can incorporate patient specific patterns of respiratory motion as well as the impact of organ motion due to respiration. For example, based on the determined respiratory motion, individualized treatment plans can be generated. The respiratory motion can also be used for more accurate tracking of the target by multi-leaf collimator (MLC), which can shape radiotherapy treatment beams. It thus can potentially reduce dose to normal tissue and the risks of secondary cancers while maintaining acceptable doses to the target.

The disclosed methods, systems, and computer-readable media are discussed with respect to a target. The target can be an organ and/or surrounding structure, and/or a tumor within an organ and/or surrounding structure, or a combination thereof. The target is discussed with respect to the prostate. It will be understood that the target is not limited to the prostate. The disclosed methods, systems, and computer-readable media may be applied to and/or include information from other targets, such as other organs and/or regions of interests, for example, breast(s), lung(s), liver, pancreas, kidney(s), as well as other organs and/or regions.

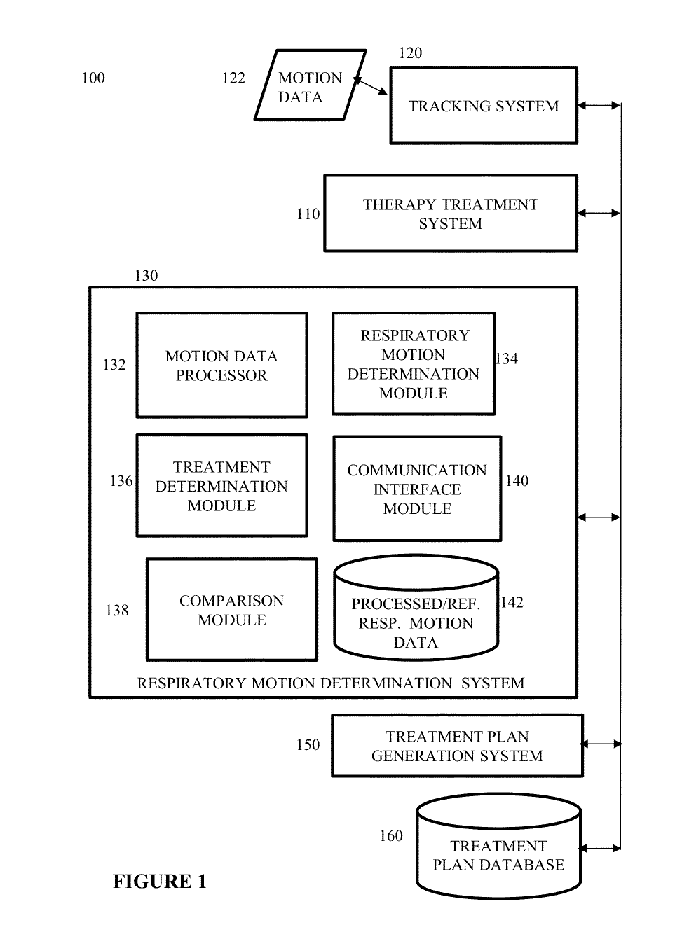

FIG. 1 shows an example of a system 100 capable of determining respiration motion from motion data of a target according to embodiments.

As shown in FIG. 1, the system 100 may include a treatment delivery system 110. The treatment delivery system 110 may be any device configured to deliver any therapeutic or diagnostic treatment, such as radiotherapy, to tissue of a target (e.g., the tissue to be treated within the patient) and to control the delivery of the treatment according to treatment parameters. For example, the treatment delivery system 110 may be any radiation treatment system. The radiation treatment system may include a radiation source (e.g., an accelerator) configured to generate radiation therapy according to treatment parameters and a multileaf Collimator (MLC) assembly configured to shape or modulate the output of the radiation source. For example, for radiotherapy, treatment delivery can generally represent any variable of a treatment, such as radiation dose, the movement of the MLC apertures, and the movement of treatment couch, collimator and/or machine gantry, or any combination thereof.

In some embodiments, the system 100 may include a motion tracking system 120. The motion tracking system 120 may be any motion tracking and localization system. In some embodiments, the motion tracking system 120 may also be an imaging guidance system. The motion tracking system 120 may be configured to track in real-time a target (e.g., a prostate) relative to a machine isocenter or another external reference frame outside of the patient to determine motion data 122 during treatment planning, set up, treatment (radiation) sessions, and/or other times of the radiation therapy process. The motion tracking system 120 may be configured to obtain the motion data 122 via any suitable technique, such as by tracking one or more internal or external markers that are positioned near the target tissue. The markers may include but are not limited to electromagnetic transponders (e.g. Calypso® 4D Localization System, available from Calypso Medical Technologies of Seattle, Wash.), which can be placed internally in the patient near or within the region of interest. The coordinates of the transponders may be determined based on the radiofrequency signal emitted by the transponders.

In some embodiments, the motion data 122 (also referred to as “organ motion data”) may be motion data of the target to be treated, for example, an organ included in the region of interest. The motion data 122 may be raw data. In some embodiments, the motion data 122 may include data representing motion of the target (x, y, and/or z) for a (time) period (t). In some embodiments, the motion data may be specific to each marker or transponder. In some embodiments, the period may correspond to at least a portion of a non-treatment planning session (e.g., pre-treatment planning or set up) and/or a treatment session (also referred to as a “fraction”). In some embodiments, one or more periods may correspond to a portion of the same session. In other embodiments, some or all of the periods may correspond to different session.

In some embodiments, the motion tracking system 120 may be any motion tracking system compatible with markers or other implanted devices. The motion tracking system 120 may include but is not limited to systems from Varian Medical Systems (e.g., Calypso), Siemens Healthcare, Accuray, RadiaDyne, Eleckta AB, as well as those developed by single modality or combinations of kV, MV, optical, MRI based tracking devices, radioactive transponders, or other systems.

In some embodiments, the markers or transponders and/or the motion tracking system 1220 may dictate the frequency at which the motion data is obtained. In some embodiments, the frequency may be about 10 Hz. In other embodiments, the frequency may be different.

In some embodiments, the system 100 may include a respiratory motion determination system 130. The treatment margin determination system 130 may be configured to determine respiratory motion of a patient from motion data of a target.

In some embodiments, the respiratory motion determination system 130 may include a motion data processor 132 configured to process the motion data 122 received from each marker. The motion data processor 132 may be configured to process raw motion data to determine the isocenter for one or more of the axes of motion (e.g., x, y, and/or z axes) for each transponder or marker. In some embodiments, the motion data processor 132 may be configured to determine the isocenter for one or more of the axes. The x axis may correspond to the right-left motion; the y axis may correspond to the anterior-posterior motion; and the z axis may correspond to the superior-inferior motion. In some embodiments, the processed motion data may include time (seconds) and right-left motion, anterior-posterior motion and/or superior-inferior motion for the isocenter for each transponder or marker or all transponders and markers

In some embodiments, the treatment margin determination system may include a respiratory motion determination module 134 configured to determine the respiratory motion (also referred to as “predicted motion data”) from the processed motion data. In some embodiments, the respiratory motion determination module 134 may be configured to at least one respiratory motion parameter associated with respiratory motion. The at least one respiratory motion parameter may be relative to one or more axes. The at least one respiratory motion parameter may include any parameter associated with displacement of the target specific to respiratory motion. In some embodiments, the at least one respiratory motion parameter may include but is not limited to maximum respiratory range of motion, average respiratory range of motion, power density, respiratory rate, among others, or a combination thereof.

In some embodiments, the system 130 may include a treatment determination module 136 configured to modify and/or adjust the treatment based on the determined respiratory motion. In some embodiments, the treatment determination module 136 may be configured to determine individualized treatment planning margins based on the predicted respiratory motion data for the one or more axes. The margins may be used by, for example, a treatment plan generation system 150 to generate a treatment plan for a patient.

In some embodiments, the treatment determination module 136 may cause the therapy treatment system 110 to track and/or gate according to the real-time respiratory motion and/or predicted respiratory motion.

In some embodiments, the system 130 may include a treatment comparison module 138. In some embodiments, the treatment comparison module 138 may be configured to compare real-time respiratory motion during a session to the determined treatment margins and/or reference respiratory motion. In some embodiments, the treatment comparison module 138 may be configured to compare respiratory motion parameter(s) to a reference respiratory motion parameter(s) and/or determined treatment margins. In some embodiments, the comparison may be based on respiratory motion parameter(s) determined based on substantially real-time respiratory motion. The treatment comparison module 138 may be configured to provide feedback based on the comparison. The treatment comparison module 138 may be configured to alert the practitioner when the respiratory motion of the target during a session is outside of the margins, for example, by a certain threshold. In this way, the treatment comparison module 138 may also be configured to determine when the patient is experiencing respiratory distress during the session.

In some embodiments, the system 130 may include a memory, for example, a database, 140 configured to store the processed respiratory motion data, reference respiratory motion and/or treatment margins for each axis for one or more patients. The processed respiratory motion data may include predicted respiratory motion data. The memory 140 may be configured to store the processed respiratory motion data and/or determined treatment margins for that patient. The memory 140 may also be configured to store a treatment margin reference that includes processed respiratory motion data and/or determined margins. In some embodiments, the treatment margin reference may be based on collected respiratory motion data for at least one patient.

In some embodiments, the treatment comparison module 138 may be configured to compare the substantially real-time respiratory motion of the target to the stored determined margins and/or processed respiratory motion data for that patient and/or to a treatment margin reference.

In some embodiments, the system 130 may include a communication interface module 142 configured to conduct receiving and transmitting of data between the modules (or systems) on the system and/or network. The communication interface module 142 may be a wired and/or wireless interface, a switched circuit wireless interface, a network of data processing devices, such as LAN, WAN, the internet, or combination thereof. The communication interface may be configured to execute various communication protocols, such as Bluetooth, wireless, and Ethernet, in order to establish and maintain communication with at least another module on the network.

In some embodiments, the system 100 may include a treatment plan generation system 150 configured to determine radiation doses to the target (e.g., prostate) and other surrounding normal structures based on the determined patient specific treatment planning margins. The treatment plan generation system 150 may be any treatment plan generation system 150. The treatment plan generation system 150, for example, may include but is not limited to Pinnacle, Eclipse, as well as others.

In some embodiments, the system 100 may include a treatment plan database 160. The treatment plan database 160 may be configured to store the treatment plans generated by the treatment plan generation system 150. The radiation therapy treatment system 110 and/or the tracking system 120 may be configured to use the respiratory motion determined by the respiratory motion determination system 130 to control the treatment and/or tracking of a target (e.g., the prostate).

In some embodiments, the system 100 may include a different set of systems or modules, including additional systems or modules, including fewer systems or modules, or sets in which the functionality of the systems or modules is divided or consolidated.

In some embodiments, the modules and/or systems of the system 100 may be connected to a data network, a wireless network, or any combination thereof. In some embodiments, any of the modules and/or systems of the system 100 may be at least in part be based on cloud computing architecture. In some embodiments, the modules and/or systems may be applied to a self-hosted private cloud based architecture, a dedicated public cloud, a partner-hosted private cloud, as well as any cloud based computing architecture.

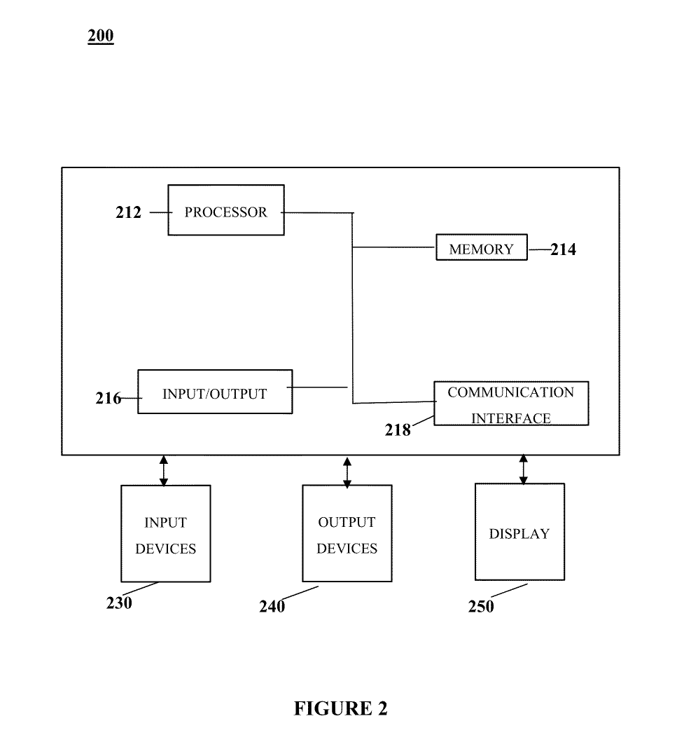

One or more of the modules and/or systems of system 100 may be and/or include a computer system and/or device. FIG. 2 is a block diagram showing a computer system 200. The modules of the computer system 200 may be included in at least some of the systems and/or modules, as well as other devices of system 100.

The systems may include any number of modules that communicate with other through electrical or data connections (not shown). In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radiofrequency network, or another similarly functioning wireless network.

It is also to be understood that the systems may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the systems may be time synchronized. In further embodiments, the systems may be time synchronized with other systems, such as those systems that may be on the medical facility network.

The system 200 may be a computing system, such as a workstation, computer, or the like. The system 200 may include one or more processors 212. The processor(s) 212 (also referred to as central processing units, or CPUs) may be any known central processing unit, a processor, or a microprocessor. The CPU 212 may be coupled directly or indirectly to one or more computer-readable storage media (e.g., memory) 214. The memory 214 may include random access memory (RAM), read only memory (ROM), disk drive, tape drive, etc., or a combinations thereof. The memory 214 may be configured to store programs and data, including data structures. In some embodiments, the memory 214 may also include a frame buffer for storing data arrays.

The CPU 212 may be configured to determine individualized treatment margins. In some embodiments, the CPU 212 may be capable of performing the data processing and/or generation of treatment plan. In other embodiments, the system may include a separate CPU for performing the data processing and/or generation of treatment plan.

In some embodiments, another computer system may assume the data analysis or other functions of the CPU 212. In response to commands received from the input device, the programs or data stored in the memory 214 may be archived in long term storage or may be further processed by the processor and presented on a display.

In some embodiments, the system 210 may include a communication interface 218 configured to conduct receiving and transmitting of data between other modules on the system and/or network. The communication interface 218 may be a wired and/or wireless interface, a switched circuit wireless interface, a network of data processing devices, such as LAN, WAN, the internet, or combination thereof. The communication interface may be configured to execute various communication protocols, such as Bluetooth, wireless, and Ethernet, in order to establish and maintain communication with at least another module on the network.

In some embodiments, the system 210 may include an input/output interface 216 configured for receiving information from one or more input devices 230 (e.g., a keyboard, a mouse, and the like) and/or conveying information to one or more output devices 240 (e.g., a printer, a CD writer, a DVD writer, portable flash memory, etc.). In some embodiments, the one or more input devices 230 may configured to control, for example, the generation of the margins and/or treatment plan, display of the margins and/or treatment plan on a display 250, printing of the margins and/or treatment plan by a printer interface, among other things.

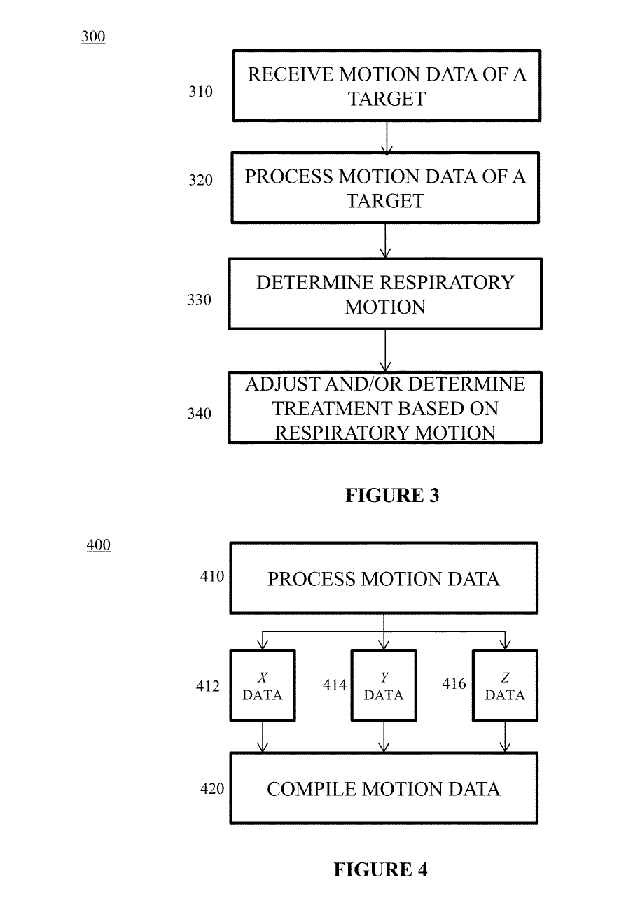

FIG. 3 illustrates a method 300 for determining respiratory motion from motion data of a target to be treated according to embodiments. In some embodiments, the method 300 may adjust and/or determine a treatment based on the determined respiratory motion. The system for carrying out the embodiments of the methods disclosed herein is not limited to the systems shown in FIGS. 1 and 2. Other systems may be used.

The methods of the disclosure are not limited to the steps described herein. The steps may be individually modified or omitted, as well as additional steps may be added. It will be also understood that at least some of the steps may be performed in parallel.

Unless stated otherwise as apparent from the following discussion, it will be appreciated that terms such as “sampling,” “averaging,” “combining,” “comparing,” “generating,” “determining,” “obtaining,” “processing,” “computing,” “selecting,” “receiving,” “summing,” “estimating,” “calculating,” “quantifying,” “outputting,” “acquiring,” “analyzing,” “approximating,” “continuing,” “resuming,” “using,” “halting,” “filtering” “evaluating,” “alerting,” “sorting,” “predicting,” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

As shown in FIG. 3, the method 300 may include a step 310 of receiving motion data of a target to be treated, for example by the treatment delivery system 110, for at least one time period. The motion data may be acquired from at least one marker or a transponder, such as an electromagnetic transponder implanted in a patient, for example, by the tracking system 120. The time period may correspond to any time period during a non-treatment session (e.g., pre-treatment planning and/or setup) or a treatment session (e.g., a fraction (f)). In some embodiments, a period may correspond to a portion of a session and/or substantially the entire session.

The motion data may include motion data for more than one marker for a period. In some embodiments, the motion data may include motion data for at least three markers for a period. In some embodiments, the motion data may include motion data for a different number of markers for each period. In some embodiments, the motion data may be received for one period. In some embodiments, the motion data may be received for more than one period. In some embodiments, the motion data may be received for at least two periods, three periods, four periods, five periods, or more than five periods.

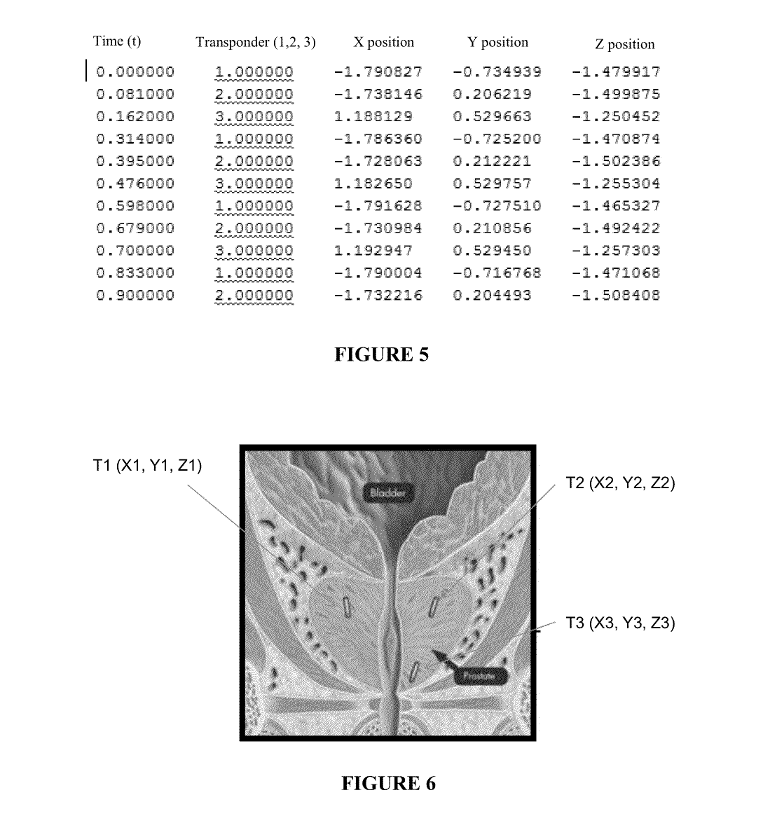

In some embodiments, the motion data may be processed. In some embodiments, the motion data may be raw. The raw motion data may include data representing the motion of the target during a period in x axis (X), y axis (Y), z axis (Z), and/or time (t). FIG. 5 shows an example of raw data obtained from an electromagnetic responder. In some embodiments, the motion data may be received in substantially real-time from the marker(s) during a session.

In some embodiments, method 320 may include a step of processing the (raw) motion data to determine the position of the target for each time point during a period, for example, by the respiratory motion determination system 130. In some embodiments, the motion data may be processed by the method shown in FIG. 4. It will be understood that other methods may be used.

As shown in FIG. 4, the method 400 may include a step 410 of processing motion data for each implanted marker for a period. In some embodiments, the data may be processed to determine the position of the target for each transponder or marker (Tᵢ) in each axis at a time point(t) during a given period (f) (Tᵢ(t_{f}, X_{f}, Y_{f}, Z_{f}). t_{f} may correspond to a time point in seconds (e.g., at 10 Hz frequency) for a period, f. X_{f}, Y_{f}, Z_{f} may correspond to position data of the target at time point (t_{f}). X_{f} may correspond to the isocenter position in right-left direction at that time point (t_{f}), f; Y_{f} may correspond to isocenter position in anterior-posterior direction at that time point (t_{f}); Z_{f} may correspond to isocenter position in superior-inferior direction at that time point (t_{f}); i may correspond to the number of markers or transponders (e.g., for 3 transponders i=1 to 3).

FIG. 4 illustrates that the data is processed for each of the axes. However, it will be understood that the data may not be processed for all of the axes. The data may be processed in one or two of the axes (at least one of the axes). In some embodiments, the axes processed may be dependent on the target. For example, some targets may have substantially limited motion in one or more of the axes.

In some embodiments, the data may be received from more than one transponder. After the data is processed into at least one of x, y, and/or z axes (steps 412, 414, and 416, respectively), the method 400 may include a step 420 of compiling the motion data received from all transponders for a period. In some embodiments, the step 420 may include processing the position data of a target (X_{f}, Y_{f}, and/or Z_{f}) obtained from each transponder for at least one axis for one or more time points (t_{f}) of that period (f) to determine the isocenter for example, by the motion data processor 132 to determine the isocenter. The isocenter for each axis may include center of mass data for each time point (t_{f}) of that period. The time point (t_{f}) may correspond to the time point at which the markers measure the position of the target. The number of time points (t_{f}) in each time period may be based on the frequency associated with the motion data obtained. The data received from all markers or transponders may be combined (e.g., averaged) to determine the center of mass (i.e., isocenter) of the target at a time point during that period. The isocenter of a target from all markers or transponders for one or more time points of the period may correspond to COMₓ, COM_{y}, and/or COM_{z}. COMₓ, COM_{y}, and COM_{z} corresponds to the average of the position in x, y, and z axes, respectively, for each transponder or marker for that time point of that period. FIG. 6 shows an example of a diagram of a target with three implanted transponders and the corresponding determined isocenters for a time point. The equation for determining the isocenter of a target for the respective axis may be determined based on the following:

COMₓ=(X₁+X₂+X₃)/3

COM_{y}=(Y₁+Y₂+Y₃)3

COM_{z}=(Z₁+Z₂+Z₃)/3 (1)

This equation (1) is based on 3 transponders or markers. However, it will be understood that more or less transponders or markers may be used and that the equation may be modified accordingly. It will also be understood that this step may be omitted, for example, if data is received from one transponder or marker.

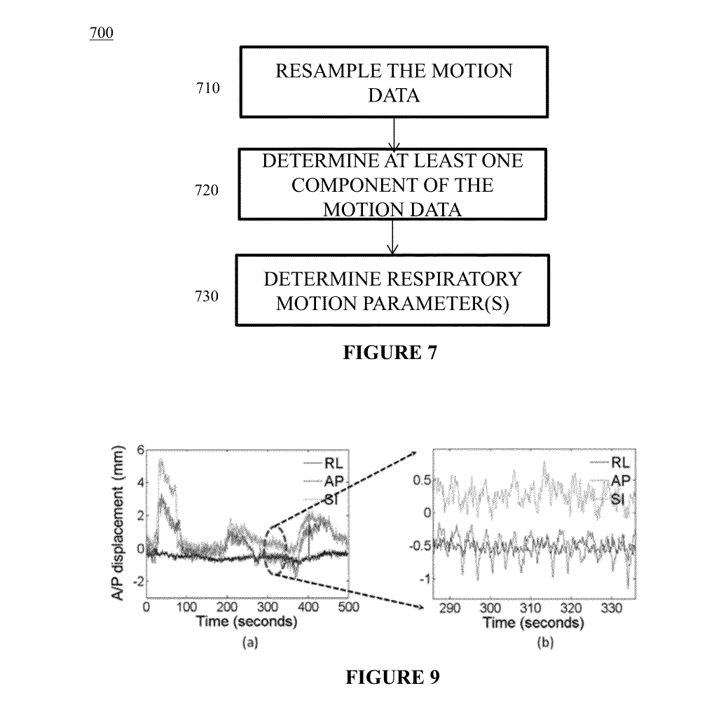

In some embodiments, the method 300 may include a step 320 of determining respiratory motion from the motion data of a target. In some embodiments, the step 320 may include determining at least one parameter associated with respiratory motion. In some embodiments, the respiratory motion may be determined by method 700 shown in FIG. 7. In other embodiments, the respiratory motion may be determined according to other methods.

As shown in FIG. 7, the method 700 may include a step 710 of resampling the motion data for each axis for a given time period. The motion data may be processed for each axis processed in FIG. 4. The step 710 can address the inconsistent intervals between time points. For examples, the time interval between each time point may not be, at 0.1 second (e.g., 10 Hz). In this way, for example, by resampling the position data, the time point and position data for a given period and axis may be uniform.

In some embodiments, the step 810 may include applying a linear interpolation to the position data at each time point collected during a period for each axis (x, y, z) (processed in FIG. 4).

In some embodiments, the resampled position data (x) for a given time point (t) may be determined based on the following:

$$x = {x}_{t 1} + ( {x}_{t 2} - {x}_{t 1} ) * \frac{( t - {t}_{1} )}{( {t}_{2} - {t}_{1} )} \qquad ( 2 )$$

For example, to get an position data (x) at time point (t), “xₜ₁” represents a position of the target (e.g., prostate) in three dimensional space for a given time point (t₁) as measured by the marker(s) and/or the tracking module 120 and “xₜ₂” represents a position of the target (e.g., prostate) in three dimensional space for the next given time point (t₂) as measured by the marker(s) and/or the tracking module 120. In some embodiments, the position data for each time point for an axis may be resampled so that the position data is provided at every 0.1 second frequency (e.g., time points are separated by 0.1 second).

The resampling is described with respect to the x axis, as an example. This step relates to resampling the motion for any axis. The following can be repeated for any of the other axes (e.g., y axis and/or z axis) or performed for any axis.

In other embodiments, the motion data may be processed according to other methods and/or omitted.

Next, the method 700 may include a step 720 of determining at least one component of the motion data. A component of the motion data means a subset of the motion data having a discrete value and/or a range of values. In some embodiments, the value and/or range of values are defined by frequency.

In some embodiments, the step 720 may include applying at least one threshold to the motion data. In this way, the motion data may be filtered into at least one component. The threshold may correspond to a value and/or range of values. In some embodiments, the value and/or range of values may correspond to a time or frequency and/or a range of time and/or frequency. In some embodiments, the threshold may correspond to a range and/or value of respiratory frequency according to clinical standards. The clinical standards, for example, may be based on age, gender, etc. of the patient. For example, the respiratory frequency (threshold) may be any range of frequencies between about 10 to 24 times per minute, which is about 0.166-0.4 Hz. A normal breathing period can be generally about 10 to 24 breaths per minute. Frequency in Hertz (Hz) can be determined by taking the inverse of the time in seconds (1/second). For example, a typical breathing rate of about 10 breaths per 60 seconds (1 minute) would correspond to about 0.166 breath per second (0.166 Hz).

In some embodiments, the respiratory frequency (threshold) may be different. In some embodiments, the respiratory frequency (threshold) may be specific to the individual patient. In other embodiments, the respiratory frequency (threshold) may also include breathing irregularities, such as anxiety and respiratory distress. For example, the respiratory frequencies may include and/or be specific to a range of frequencies considered to correspond to anxiety and/or respiratory distress. For example, in some embodiments, the respiratory frequency may be any range of frequencies between about 0.15-0.7 Hz, which generally includes about 10 to 40 breaths per minute. In other embodiments, the respiratory frequency may be above or below that range. For example, the respiratory frequency may be in any range of frequencies between about 0.05-1.0 Hz.

In some embodiments, any number of thresholds corresponding to different respiratory frequencies (or range of respiratory frequencies) may be applied to determine more than one component. For example, at least two different thresholds corresponding to different respiratory frequencies (or range of respiratory frequencies) may be applied to determine two different components, for example, one that corresponds to normal respiratory frequency and one that corresponds to irregular respiratory frequency.

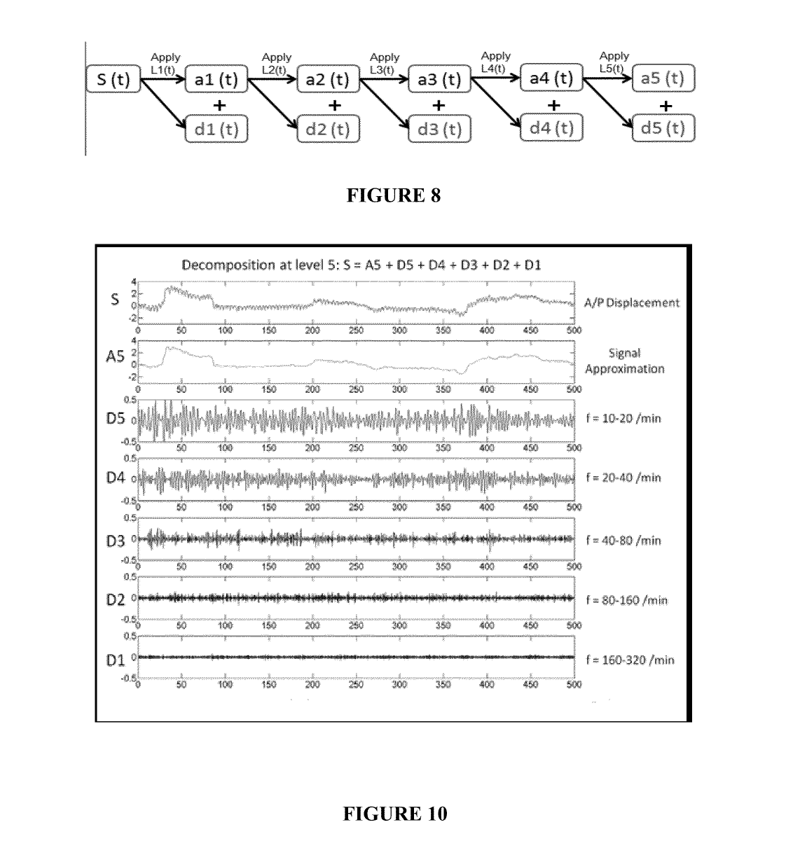

FIG. 8 shows an exemplary diagram of determining at least one component of the motion data according to some embodiments. In this example, the motion data can be filtered using a plurality thresholds to determine a plurality of components. The motion data is shown as the amount of displacement in mm with respect to time in seconds. As shown in FIG. 8, S(t) corresponds to the motion data of a target for one axis for a period. d1(t), d2(t), d3(t), d4(t), and d5(t) correspond to components of the motion data. L1(t), L2(t), L3(t), L4(t) and L5(t) correspond to different thresholds. a1(t), a2(t), a3(t), a4(t), and a5(t) correspond to filtered motion data.

It will be understood FIG. 8 is an example and that the number of components and threshold values are not limiting. Any number of components may be determined and any number of thresholds may be applied. In this way, more or less components may be determined by applying more or less thresholds. For example, one or more of the components shown in FIG. 8 may be combined, omitted, and/or a combination thereof. Additionally, one or more components may be added to any combination of the components shown in FIG. 8. Additionally, the thresholds are not limited to those shown in FIG. 8 and different thresholds may be used.

For example, in some embodiments, the determining step may include determining a component of the motion data having a range of d4(t)-d5(t) by applying a threshold having a range from L(4)t-L5(t). In another example, the determining step may include determining a first component of data corresponding to d4(t) and determining a second component of data corresponding to d5(t).

FIG. 9 shows an example of a motion data in the right-left (x), anterior-posterior (y), and superior-inferior (SI) obtained from a tracker and processed according to FIG. 4. FIG. 10 shows an example of motion data in the anterior-posterior (y) direction filtered using the diagram shown in FIG. 8. The motion data (S(t)) corresponds to motion data in the y-axis (anterior-posterior). In this example, L1(t) corresponds to a threshold value of about 2.5 Hz (0.4 seconds per breath), L2(t) corresponds to about 1.25 Hz (0.8 seconds per breath), L3(t) corresponds to about 0.625 Hz (1.6 seconds per breath), L4(t) corresponds to about 0.3125 Hz (3.2 seconds per breath), and L5(t) corresponds to about 0.1563 Hz (6.39 seconds per breath). It will be understood that the thresholds are not limited to these values and may have different values. By applying these thresholds to the motion data (S1(t)), D1 can correspond to a component of motion data having a range of about 2.5-5 Hz; D2 can correspond to a component of motion data having a range of about 1.25-2.5 Hz; D3 can correspond to a component of motion data having a range of about 0.625-1.25 Hz; D4 can correspond to a component of motion data having a range of about 0.3125-0.625 Hz; and D5 can correspond to a component of motion data having a range of about 0.1563-0.3125 Hz. In this way, the filtered motion data A1(t), A2(t), A3(t), A4(t) can correspond as follows: A1(t)=A2(t)+D2(t); A2(t)=a3(t)+d3(t); A3(t)=d4(t)+A4(t); A4(t)=A5(t)+d5(t). Also, S(t)=A1(t)+d1(t).

After at least one component is determined, the method 300 may include a step 330 of determining respiratory motion. In some embodiments, the step 330 may include determining at least one respiratory motion parameter.

In some embodiments, the respiratory motion may be determined based on at least the one component of motion data. In some embodiments, the respiratory motion may be determined based on one or more components of motion data to be within a discrete range and/or value. In some embodiments, the respiratory motion may be determined based solely on the components determined. In other embodiments, the respiratory motion may be determined based on one or more of the components determined. For example, one or more components determined may be discarded or omitted from the determining step 330. These one or more components may be determined to be noise, background physiological parameters, etc., for example, based on the frequency.

In the example shown in FIG. 10, components D1-D3 can be discarded because it is likely noise, background physiological parameters, etc. D1-D3 can be considered to have a frequency ranges that are too high for respiratory motion. A normal respiratory motion may be considered to be about 10 to 24 breaths per minute. D1 corresponds to motion data having a respiratory frequency of about 160-320 times per minute; D2 corresponds to motion data having a respiratory frequency of about 80 to 160 times per minute; and D3 correspond to motion data having respiratory frequency of about 40 to 80 times per minute. In this example, the respiratory motion can be considered from D4, which corresponds to motion data having respiratory frequency of about 20-40/min, and D5, which corresponds to motion data having respiratory frequency of about 10-20/min. D4 may include respiratory frequency resulting from anxiety and/or respiratory distress.

In some embodiments, the method 700 may include a step of 730 determining respiratory motion from the at least one component of motion data. The step 730 may include determining at least one respiratory motion parameter. The step 730 may include quantifying the respiratory motion from the at least one component of motion data for at least one axis to determine the at least one respiratory motion parameter. The quantifying may include quantifying at least one attribute of the component. The at least one attribute may include but is not limited to amplitude, frequency, among others, or a combination thereof. In some embodiments, the at least one respiratory motion parameter may include any parameter associated with displacement of the target specific to respiratory motion. The at least one respiratory motion parameter may include but is not limited to respiratory rate, maximum respiratory range of motion, average respiratory range of motion, power density, among others, or a combination thereof. These parameters may be determined for at least one component for at least one axis according to any method. In this way, by determining parameters associated with the displacement of the target specific to respiratory motion, the impact of the respiratory motion may be determined.

In some embodiments, the quantifying may be based on at least one component for a period for at one axis. In other embodiments, the quantifying may be based on at least one component for a period for at least two axes. In some embodiments, for example, the axes may include the y axis (AP direction) and z axis (SI direction). The at least one component having a frequency including at least the standard respiratory frequency. In the example, FIG. 10, the respiratory motion associated with D4 plus D5 components for these axes may be quantified. Next, the amplitude from the respiratory motion may be determined. The amplitude (RM) for two axes may be determined from the following

$$\sqrt{\text{?} + \text{?}} . \\ \text{?} \text{indicates text missing or illegible when filed} \qquad ( 3 )$$

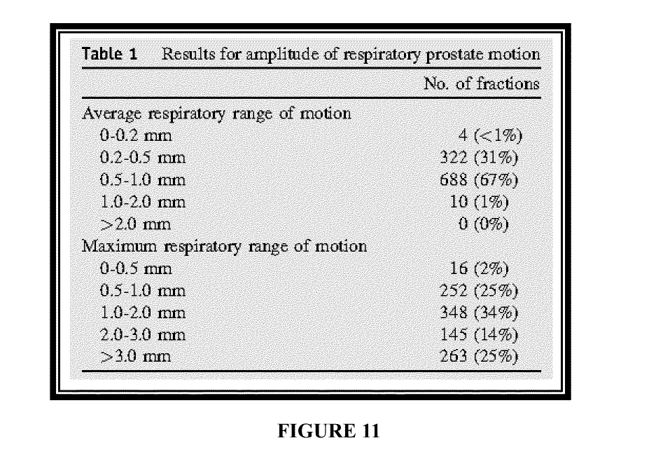

In some embodiments, the average respiratory range of motion and/or maximum respiratory range may be determined. The average respiratory range of motion may be determined as 4×R_{Mmeans}. The average absolute amplitude (R_{Mmeans}) may be determined from the determined amplitude. The average respiratory range of motion may take into account the positive and negative signs associated with the displacement. For example, an oscillation with an amplitude of above 2 mm (±1 mm) would have an average absolute amplitude of about 0.5 mm. The maximum respiratory range (R_{Mmax}) may be determined by determining the difference between the maximum and minimum amplitude. FIG. 11 shows of quantified respiratory motion from at least the motion data shown in FIG. 10.

In some embodiments, the method 300 may include a step of outputting the respiratory motion. In some embodiments, the outputting may include but is not limited to displaying the respiratory motion (e.g., the respiratory motion parameters), printing the respiratory motion, and storing the respiratory motion remotely or locally, e.g., in memory 160. In other embodiments, the respiratory motion may be forwarded for further processing. In some embodiments, the method may further include transmitting the respiratory motion to another system, for example, the radiation therapy treatment system 110 and/or tracking system 120.

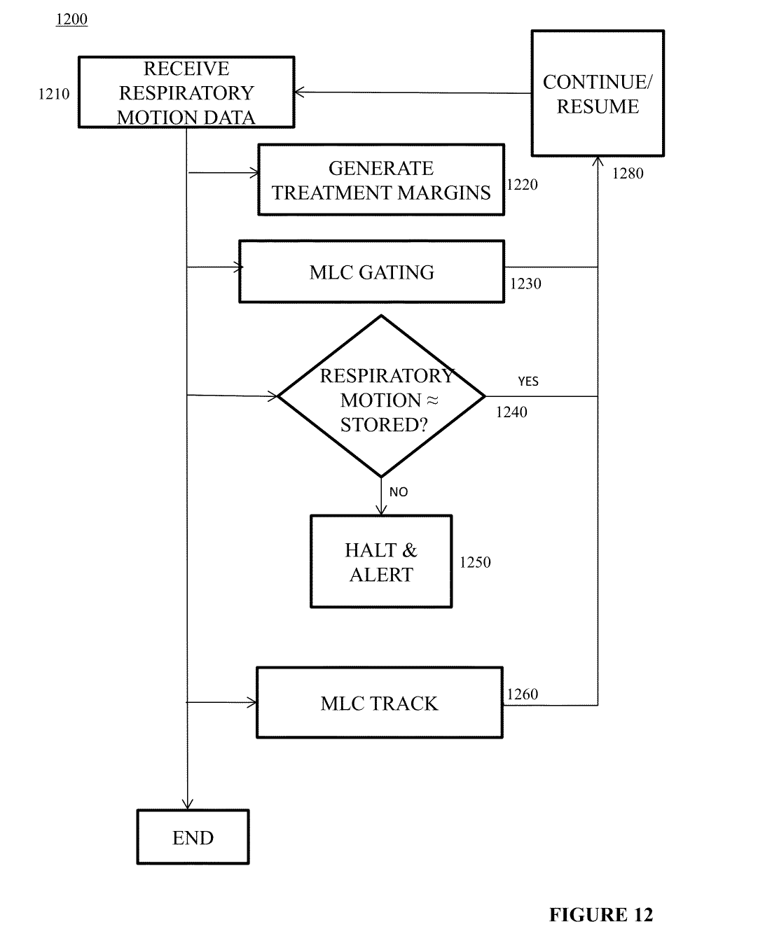

Next, the method 300 may include a step 340 of adjusting and/or determining treatment based on the determined respiratory motion. In some embodiments, the step 340 may be based on a comparison of a determined respiratory motion parameter to a reference respiratory motion parameter. The reference respiratory motion parameter may be according to clinical standards and/or specific to that patient. In some embodiments, the respiratory motion parameter may be determined substantially in-real time based on motion data obtained substantially in real-time. FIG. 12 shows examples of how treatment may be adjusted and/or determined based on the respiratory motion.

As shown in FIG. 12, the method 1200 may include a step 1210 of receiving the respiratory motion data (e.g., the respiratory motion parameters). For example, the motion data may be transmitted from the respiratory motion determination system 130 to another system shown in FIG. 1 or within the respiratory motion determination system 130. In some embodiments, the method 1200 may include any one or any combination of: a step 1220 of generating treatment margins, a step 1230 of MLC gating, a step of 1240 of evaluating respiratory motion and/or a step 1260 of MLC tracking. In some embodiments, one or more of these steps may be performed in substantially real-time.

In some embodiments, the step 1220 may include generating treatment margins based on the respiratory motion data. In some embodiments, a treatment plan based on the determined treatment margins. In some embodiments, the step 1220 may be according to any known methods.

In some embodiments, the step 1230 of MLC gating and/or step 1260 of MLC tracking may be based on the respiratory motion. In some embodiments, the MLC gating and/or MLC tracking may be based on respiratory motion in real-time for a time point ti. In some embodiments, the MLC gating and/or MLC tracking may be based on predicted motion of the target determined from the respiratory motion. In this way, the MLC can track the target and deliver a treatment with a margin taking into account respiratory motion.

In some embodiments, the respiratory motion may be used to provide feedback during a session, for example, a radiation treatment session and/or non-treatment session. In some embodiments, the step 1240 may include comparing the respiratory motion to reference frequency for a normal patient according to clinical standards and/or a reference frequency for that patient. In this way, respiratory distress during a session may be determined. If the respiratory motion is not within the reference frequency (NO at step 1240), then the practitioner may be alerted and/or treatment may be halted. If the respiratory motion is within the reference frequency (YES at step 1240), then the treatment and/or imaging may continue.

In some embodiments, the step 1240 may include comparing the real-time respiratory motion data to the stored treatment planning margins and/or the corresponding stored predicted respiratory motion data, for example, by the comparison module 138. In some embodiments, the treatment planning margins and/or predicted respiratory motion data may be stored in the memory 140 and/or treatment plan database. In some embodiments, the stored treatment planning margins and/or posterior distribution may include the treatment planning margins and/or predicted respiratory motion data determined for the patient based on the respiratory motion data. In other embodiments, the stored treatment planning margins and/or predicted respiratory motion data may be from a reference treatment planning margins and/or predicted respiratory motion data.

If it is determined that the real-time motion data representing respiratory motion of the patient is outside of the stored margins and/or the predicted respiratory motion by a threshold (NO at step 1240), then the practitioner may be alerted and/or treatment may be halted. The threshold may depend on clinical circumstances and the comparison. For example, the threshold for the margins may be about 1 or 2 mm The thresholds for margins and the predicted motion are not limited to these values and may be any value.

The respiratory motion data may be outside the range due to errors, for example, resulting from human errors (e.g., positioning errors, wrong patient, etc.), treatment planning and/or treatment execution errors, among others. The treatment may resume (step 1280) after the errors have been addressed. If the respiratory motion data is within the range (YES at step 1240), the treatment and/or imaging may continue (step 560). The monitoring (e.g., steps 1210, 1230-1260 and 1280) of the respiratory motion data may be repeated over at least a portion of the session. For example, the monitoring may be terminated at a point and/or the end of a session.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

In some embodiments, the disclosed methods (e.g., FIGS. 3, 4, 7, and 12) may be implemented using software applications that are stored in a memory and executed by a processor (e.g., CPU) provided on the system 100. In some embodiments, the disclosed methods may be implanted using software applications that are stored in memories and executed by CPUs distributed across the system 100. As such, any of the systems and/or modules of the system 100 may be a general purpose computer system, such as system 200, that becomes a specific purpose computer system when executing the routine of the disclosure. The systems and/or modules of the system 100 may also include an operating system and micro instruction code. The various processes and functions described herein may either be part of the micro instruction code or part of the application program or routine (or combination thereof) that is executed via the operating system.

If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure. An example of hardware for performing the described functions is shown in FIGS. 1 and 2.

It is to be further understood that, because some of the constituent system components and method steps depicted in the accompanying figures can be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. A method of determining an impact of respiratory motion with respect to movement of a target to be treated, comprising:
processing motion data of a target to be treated obtained from at least one marker for at least one period, each period including a plurality of time intervals;
determining at least one component of the motion data in at least one axis of motion, the at least component corresponding to a subset of the motion data having a discrete value and/or a range of values; and
determining respiratory motion from at least one component of the motion data.

2. The method according to claim 1, wherein the determining the respiratory motion includes determining at least one respiratory motion parameter associated with displacement of the target specific to respiratory motion.

3. The method according to claim 2, wherein the at least one respiratory motion parameter includes at least one of maximum respiratory range of motion, average respiratory range of motion, respiratory rate, and/or power density.

4. The method according to claim 2, wherein the determining the at least one respiratory motion parameter includes quantifying at least one attribute of the at least one component in at least two axes.

5. The method according to claim 1, wherein the determining the at least one component includes applying at least one threshold to the motion data.

6. The method according to claim 5, wherein the threshold corresponds to a range of frequencies associated with a respiratory pattern.

7. The method according to claim 6, wherein the threshold corresponds to a range of frequencies of about 0.166-0.4 Hz.

8. The method according to claim 1, further comprising:
wherein the at least one marker is an electromagnetic transponder.

9. The method according to claim 1, further comprising:
comparing the determined respiratory motion to a reference respiratory motion; and
causing treatment of the target to be adjusted based on the comparing.

10. A system for determining respiratory motion of a patient, comprising:
a motion data processor configured to process motion data of a target to be treated obtained from at least one marker for one or more periods, each period including a plurality of time intervals; and
a respiratory motion determination module configured to determine at least one component of the motion data in at least one axis of motion, the at least component corresponding to a subset of the motion data having a discrete value and/or a range of values and configured to respiratory motion from at least one component of the motion data.

11. The system according to claim 10, wherein the respiratory motion determination module is configured to determine at least one respiratory motion parameter associated with displacement of the target specific to respiratory motion.

12. The system according to claim 11, wherein the at least one respiratory motion parameter includes at least one of maximum respiratory range of motion, average respiratory range of motion, respiratory rate, and/or power density.

13. The system according to claim 12, wherein the respiratory motion determination module is configured is to quantify at least one attribute of the at least one component in at least two axes and the respiratory motion parameter is based on the at least one attribute.

14. The system according to claim 10, wherein the respiratory motion determination module is configured to determine the at least one component by applying at least one threshold to the motion data.

15. The system according to claim 14, wherein the threshold corresponds to a range of frequencies associated with a respiratory pattern.

16. The system according to claim 15, wherein the threshold corresponds to a range of frequencies of about 0.166-0.4 Hz.

17. The system according to claim 10, further comprising:
wherein the at least one marker is an electromagnetic transponder.

18. The system according to claim 10, further comprising:
a comparison module configured to compare the determined respiratory motion to a reference respiratory motion and cause treatment of the target to be adjusted based on the comparing.

19. A computer-readable storage medium storing instructions for determining respiratory motion, the instructions comprising:
processing motion data of a target to be treated obtained from at least one marker for at least one period, each period including a plurality of time intervals;
determining at least one component of the motion data in at least one axis of motion, the at least component corresponding to a subset of the motion data having a discrete value and/or a range of values; and
determining respiratory motion from at least one component of the motion data.

20. The computer-readable storage medium according to claim 19,
wherein the determining the at least one component includes applying at least one threshold to the motion data.
